(19) United States
(12) Patent Application Publication
(10) Pub. No.: US 2014/0242185 A1
(43) Pub. Date: Aug. 28, 2014

(54) Method of Enhancing Health of a Person

(71) Applicant: D&Y Technologies, Holden, MO (US)

(72) Inventors: Shui Yin Lo, Pasadena, CA (US); David L. Gann, Holden, MO (US)

(73) Assignee: D&Y Technologies, Holden, MO (US)

(21) Appl. No.: 14/177,918

(22) Filed: Feb. 11, 2014

Related U.S. Application Data
(60) Provisional application No. 61/769,461, filed on Feb. 26, 2013.

(51) Int. Cl. A61K 33/00

(52) U.S. Cl. CPC A61K 33/00
USPC 424/600

(57) ABSTRACT

A method of enhancing health of a person includes administering stable water clusters to persons having an autoimmune disease, pain, a chronic disease, a mental disease, a genetic disease from malfunction of a normal DNA, being an athlete for improving his performance and alleviating soreness, suffering from overworking, stress and toxins etc., by drinking a solution containing stable water clusters, swallowing small objects which contain the stable water clusters, putting a topical cream which contains the stable water clusters on skin, breathing the stable water clusters through mouth, putting drops which contain the stable water clusters into eyes, ears or nose, cleaning colon with solution that contains the stable water clusters, eating food that contains the stable water clusters, injecting solution that contain the stable water clusters into blood vessel etc.

### CROSS REFERENCE TO A RELATED APPLICATION

This patent application claims it priority from the Provisional Patent Application Ser. No. 61/769,461 filed on Feb. 26, 2013 under 35 USC 119(a)-(d) and fully incorporates the above-identified Provisional Patent Application by reference thereto.

### BACKGROUND OF THE INVENTION

The present invention relates to method for enhancing health of people who are in need of such enhancement.

For centuries numerous substances and medications and also methods for health enhancement as well as preventing and curing diseases have been developed and used. Their listing or even classification would be so enormously long that it is believed that it would not make sense. It should be however stated that it is always advisable to develop and use new efficient methods of enhancement of heath of human being

### SUMMARY OF THE INVENTION

In the present application a new method of enhancing health of a person is proposed. In the accordance with the present invention, the method of enhancing health of a person includes administration of stable water clusters to a person.

In accordance with one feature of the invention, administration of the stable water clusters according to the invention can include administering the stable water clusters to the person having an autoimmune disease including arthritis rheumatoid arthritis, lupus, diabetes, cancer, asthma, and allergy.

In accordance with another feature of the present invention, the administration of the stable water clusters can include administering the stable water clusters to the person having pain including neck pain, upper back pain, lower back pain, pain in fingers, pain in hands, pain in arms, pain in thighs, pain in abdominal area, pain in stomach, pain in heart, pain from an accident, pain in head, pain in ear, pain in eyes, pain in nose, pain in a cheek, pain in a gum, tooth pain, pain in mouth migraine, and pain in sine.

In accordance with a further feature of the present invention, the administration of the stable water clusters can include administering of the stable water clusters to the person having a chronic disease including chronic fatigue syndrome and fibromyalgia.

Still a further feature of the present invention resides in that the administration of the stable water clusters can include administering the stable water clusters to the person having a mental disease including depression, bipolar disorder, schizophrenia ADHD ADD and ASD.

Still another feature of the present invention resides in that the administration of the stable water clusters can include administering of stable water clusters to a person having a genetic disease from malfunction of a normal DNA.

Another feature of the present invention resides in that the administration of the stable water clusters can include administering of the stable water clusters to the person who is an athlete for improving his performance and alleviating soreness after workouts.

A further feature of the present invention resides in that the administration of the stable water clusters can include administering of the stable water clusters to the person who suffers, from overworking, stress and toxins, such as biological, chemical and physical toxins.

The novel features of the present invention will be defined in the appended claims. The invention itself, however, will be best understood from the following description of the preferred embodiments which is accompanied by the following drawings.

### BRIEF DESCRIPTION OF THE DRAWINGS

FIGS. 1 and 2 are views showing two examples of thermographs of patients who were drinking ordinary distilled water and water with stable water clusters;

FIG. 3 shows a correlation between effects of distilled water and stable water clusters at temple areas;

FIG. 4 shows a correlation between effects of distilled water and stable water clusters at ear area;

FIG. 5 shows a correlation between effects of distilled water and stable water clusters at collar bone area;

FIG. 6 shows a correlation between effects of distilled water and stable water clusters at left and right eye areas;

FIG. 7 shows a correlation between effects of distilled water and stable water clusters at left and right mouth corners;

FIG. 8 shows a correlation between distilled water and stable water clusters at ten acupoints;

FIG. 9 shows a distribution of a number of independent measurements at temple acupoints;

FIG. 10 shows a distribution of a number of independent measurements at ear acupoints;

FIG. 11 shows a distribution of a number of independent measurements in a collar bone area, thyroids;

FIG. 12 shows a distribution of a number of independent measurements at left and right eyes;

FIG. 13 is a view showing a distribution of a number of independent measurements at left and right of a mouth;

FIG. 14 is a view showing a distribution of a number of independent measurements at ten points for a mouth;

FIG. 15 is a view showing a health progress over 1.5 months time period of drinking stable water clusters; and

FIGS. 16 and 17 are views showing theremoimages of a male patient and a female patient during an initial visit and a follow up visit.

### DESCRPTION OF THE PREFERRED EMBODIMENTS

The method for enhancing a health of a person in accordance with the present invention includes administration to a person of an efficient amount of stable water clusters. The administration of the stable water clusters is carried out by administering in each case a product with stable water clusters. Some of such products are disclosed in our U.S. Pat. No. 8,383,688 issued on Feb. 26, 2013 which is incorporated here by reference thereto.

The stable water clusters are solid stable water clusters as disclosed in the above identified patent. They can have a ring-shaped structure of pentagon, hexagon, rectangle, joined together ring-shaped structures linear structures, kidney-shaped structures, double-helix structures, etc. They can have nanometer sizes. The solid stable water clusters are produced by methods disclosed in our U.S. Pat. No. 8,193,251 issued on Jun. 5, 2012 which is also incorporated here by reference thereto.

In accordance with the present invention, the solid stable water clusters can be administered to a person drinking a solution containing stable water clusters through mouth, by swallowing small objects which contain the stable water clusters, by putting a topical cream which contains the stable water clusters on skin as disclosed in our U.S. Pat. No. 8,575,223. The solid stable water clusters can also be administered to a person by breathing the stable water clusters through mouth or nose, putting drops which contain the stable water clusters into eyes, ears or nose, cleaning colon with solution that contains the stable water clusters, eating food that contains the stable water clusters, and injecting solution that contain the stable water clusters into blood vessels.

In accordance with a further feature of the present invention, the solid stable water clusters can be administered to persons in the above described ways, in particular to persons having an autoimmune disease including arthritis rheumatoid arthritis, lupus, diabetes, cancer, asthma, and allergy.

The solid stable water clusters can be administered to persons having pain such as neck pain, upper back pain, lower back pain, pain in fingers, pain in hands, pain in arms, pain in thighs, pain in abdominal area, pain in stomach, pain in heart, pain from an accident, pain in head, pain in ear, pain in eyes, pain in nose, pain in a cheek, pain in a gum, tooth pain, pain in mouth migraine, and pain in sine.

Furthermore the solid stable water clusters can be administered to persons having chronic diseases including chronic fatigue syndrome and fibromyalgia or mental diseases including depression, bipolar disorder, schizophrenia ADHD ADD and ASD.

The stable water clusters can be also administered to persons having a genetic disease from malfunction of a normal DNA, to athletes for improving their performance and alleviating soreness after workouts, to persons who suffers, from overworking, stress and toxins selected from the group including biological, chemical and physical toxins.

In all above mentioned cases and in other which are not specifically mentioned, the administration of effective amounts of the stable water clusters enhances health of persons.

The enhancing of health with the use of the stable water clusters will be first described in an example of a healing effect of stable water clusters on the brain, thyroid, and others. The stable water clusters, which is a new solid phase of water that is stable in room temperature and pressure, has been presented sometime ago. The stable water clusters are made up of pure water molecules only without any other impurity, and they have permanent electric dipole moment. Stable water clusters has been proposed to be the constituents of meridian system in Chinese medicine. The yin and yang of Chinese medicine fits nicely to be the electrical and positive charges of these stable water clusters. Recently the electric fields of these stable water clusters, which are emitted from the charges of the electric dipole, have been observed via Atomic Force Microscope. A double blind study using blood peripheral cells has reported significant increase in cytokines production that enhances immune ability. Recently a pilot study has found that there is improvement in children with ASD (Autistic Spectrum Disorder) from drinking water with stable water clusters. If stable water clusters is the basic building block of meridian systems, drinking SWC may repair meridians, enhance qi to flow smoothly, enable the body to balance itself, and greatly restore its own healing ability. Its healing effect may be like that of the needles in acupuncture. One needle can cure many diseases. So we expect the stable water clusters may have healing effect on many aspects of human health. Over the past one and a half years we have more than 500 subjects where their thermographs were taken. For this study we choose to concentrate the effect of stable water clusters on the brain and thyroid for a group of 30 persons above 50 years old.

Methods and results of measuring very short term healing effect of the stable water clusters are presented below.

Volunteers above age 50 were recruited without any restriction on their health. There were 16 female and 14 male subjects with ages ranging from 53-77 years old. They joined our study for various reasons. They ranged from wanting just to have better health to people who cannot get well from any other methods and hope SWC may improve their health. For the present report a group of 30 subjects were studied and their data were analyzed.

The method contained two distinct features that were not normally done in clinical test. First, we used each subject as control. Each of the 30 subjects participated in the control group as well as in the experimental group.) The immediate healing effects of stable water clusters (SWC) were studied by using infrared image system to take thermographs before and 15 minutes after drinking 8 oz ordinary distilled water. Subsequently, through thermographs we could measure the changes of body surface temperatures that the SWC water had on each subject. The measurement of these temperatures changes of body surface became a measure of the healing effect of the ordinary distilled water, which was commonly called placebo effect, and the healing effect of SWC. Since thermographs were passive, non-intrusive device, repeated independent sets of measurements can be done many times without affecting one another.

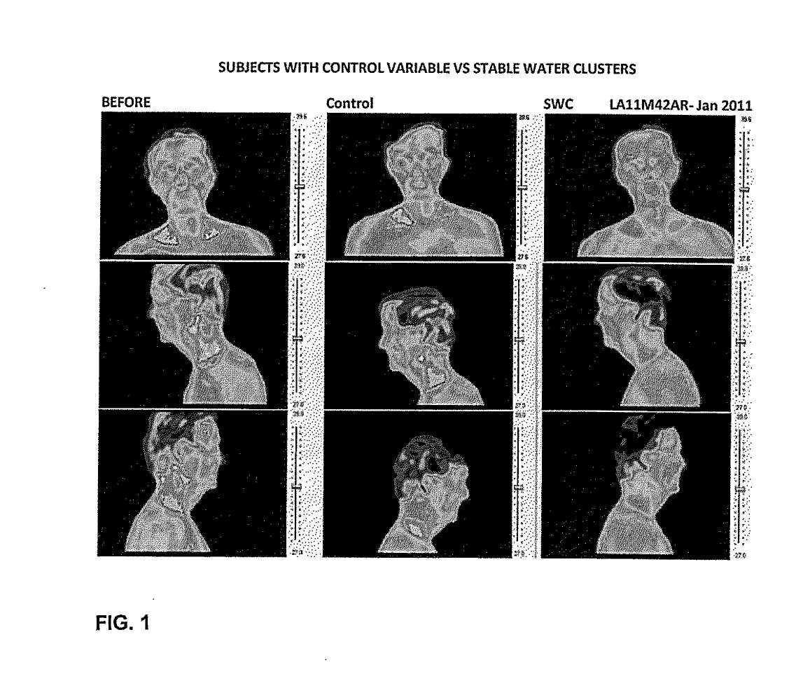

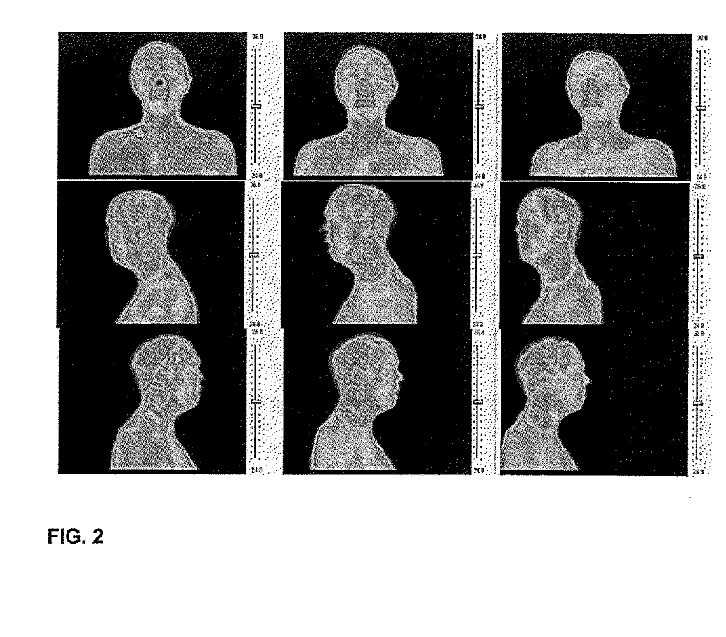

These were the procedures of our experiment. A first set of thermograph of the subject was taken where subjects did not drink any liquid. Then each subject was given an 8 ounce glass of ordinary distilled water to drink. A second set of thermograph of the subject was taken fifteen minutes afterwards. Then the subject was given 8 ounce glass of SWC to drink. A third set of thermographs was taken fifteen minutes afterwards. By comparing the first and second set of thermographs the effect of ordinary water was measured. It serves as the control for that person. Two examples are shown in FIG. 1 and FIG. 2, showing subjects with control variable vs stable waster clusters.

It is clear from these pictures there are many hot spots and hot areas. We chose to study hot areas and hot spots that could be identified with acupuncture point in Chinese medicine. In particular ten acupoints were chosen. From the thermographs of the frontal face six hot areas or hot spots were chosen: left and right BL1 (the inner extreme points in the eyes), left and right ST4 (outer corners of the mouth), and left and right ST12 (⅔ up the collar bone beneath the neck). From the thermographs of the two sides of the head, four hot areas or hot spots were chosen: left and right SJ21 (next to the frontal center of the ear lobe), and left and right GB14 (temple area). These ten hot spots were chosen because of they were present in all subjects not just in the 30 subjects we chose here, but in all 500 subjects that we studied so far.

For these hot areas and hot spots we chose to measure the maximum temperature as representing the seriousness of the health problem. The hotter the maximum temperature, the more inflamed the acupoint or the meridian, where the hot spot resided, was.

Let T₁ (Aᵢ) be the maximum temperature at ten acupoints Aᵢ, where i=1, 2, . . . , 10, before the drinking any liquid, T₂ (Aᵢ₎) the maximum temperature at acupoints Aᵢ 15 minutes after drinking ordinary distilled water, and T₃ (Aₗ) the maximum temperature at acupoints Aᵢ 15 minutes after drinking SWC. The differences in maximum temperatures are:

Δ_{2,1} (Aᵢ)=T₂ (Aᵢ)−T₁ (Aᵢ), (1)

which measures the placebo effect of drinking ordinary distilled water. A negative Δ means cooling of the hot spot, and a positive Δ means a warming up of the hot spat. The difference in maximum temperature 15 minutes after drinking SWC as compared with drinking ordinary distilled water is

Δ_{3,2} (Aᵢ)=T₃ (Aₗ)−T₂ (Aᵢ). (2)

The set of hot spots Aᵢ consists of two hot spots at the eye area BL1, two hot spots ST4 at the corners of the mouth, two hot spots ST12 in the collar bone area, two hot spots GB14 on the temples, and two hot spots SJ21 near the center of the ears. Thus, we have ten differences Δ_{2,1} (Aᵢ) of maximum temperatures at these ten hot spots that represent placebo effect caused by drinking ordinary distilled water. Furthermore, we have ten differences Δ_{3,2} (Aᵢ) of maximum temperatures that are caused by drinking additional SWC water. When we subtract Δ_{3,2} (Aᵢ) by Δ_{2,1} (Aᵢ) to get the difference

Δ(Aᵢ), this difference

Δ(Aᵢ)=Δ_{3,2} (Aᵢ)−Δ_{2,1} (Aᵢ) (3)

Or

Δ(Aᵢ)=[T₃(Aₗ)+T₁(Aᵢ)]−2 T₂ (Aᵢ). (4)

It represents the healing effect of SWC with placebo effect being subtracted out.

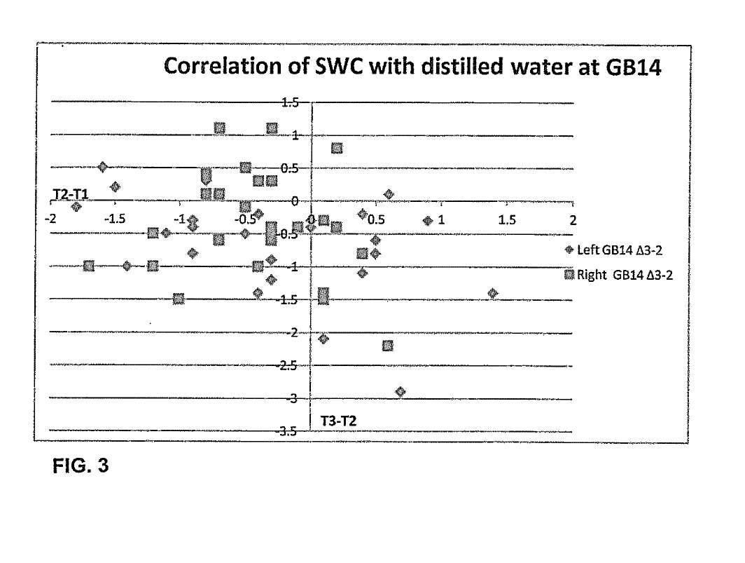

First we want to demonstrate by plotting the correlation of Δ_{2,1} with Δ_{3,2}. In FIG. 3 for each subject the changes in maximum temperatures at left and right acupoints GB 14 are shown as a point on the x-y plot with its Δ_{2,1} values in the x-axis and its Δ_{3,2} values in the y-axis. If the effect of SWC is exactly the same as the effect of distilled water, then all points should lie on a straight line. In FIG. 3 there is no discernable pattern of the points, it is a random distribution of points on the x-y plane. Therefore, it means the effect of SWC is completely different from the effect of distilled water. This lack of correlation among different points on the plot demonstrates that SWC is different from distilled water.

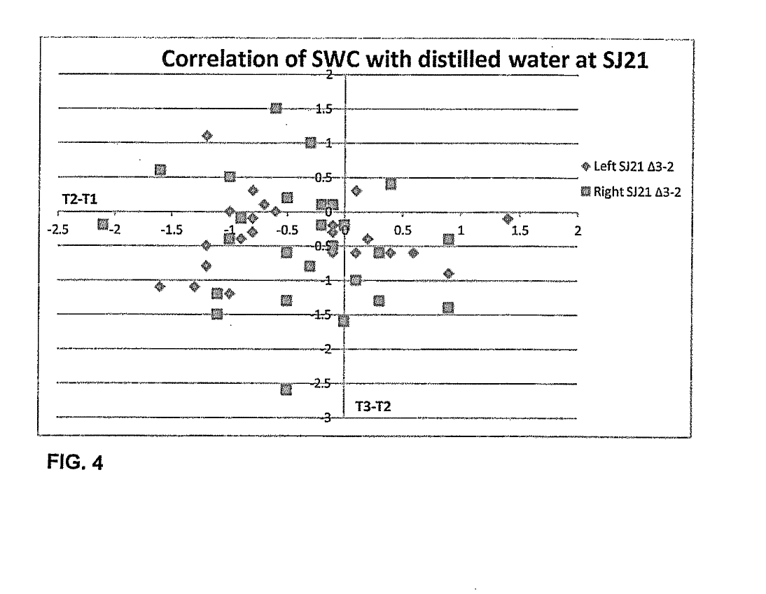

In FIG. 4, 5, 6, 7 correlations of the effect of distilled water and SWC are shown in the ear areas at SJ21, the collar bone areas at ST12, eye areas at BL1, and the mouth areas at ST4, respectively. Again, there are no discernable patterns of points on these five plots. This lack of correlation among different points on the plots indicates that SWC is different from distilled water in its healing effect.

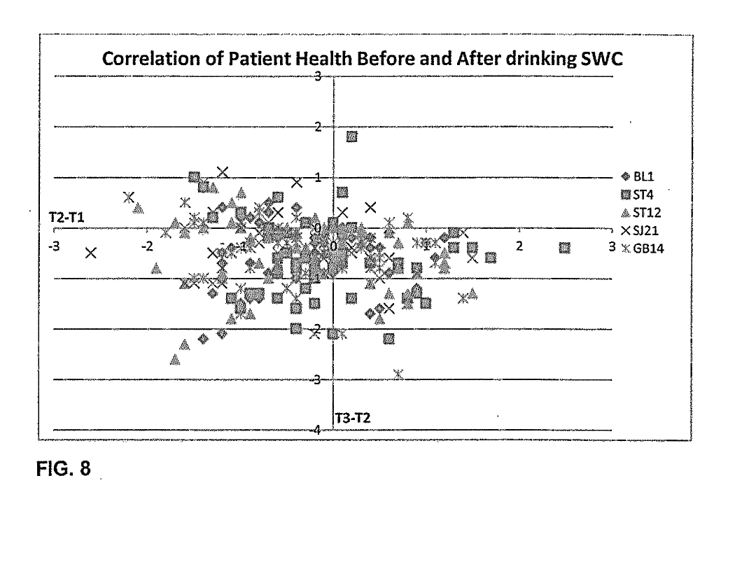

In FIG. 8 it is a correlation plot of Δ_{2,1} with Δ_{3,2} for all points in these ten areas of 30 subjects.

FIG. 3 shows the correlation between the effect of distilled water (Δ_{2,1} as x-axis) and SWC (Δ_{3,2} as y-axis) at the temple areas at both left and right GB 14. There was a total of 54 total cases with 45 of those cases having >±0.25 temperature change resulting in 83.3% significance. Positive values mean heating up and negative values mean cooling down. Note: the points that are repeated are shown as only one marker in the graph.

FIG. 4 shows the correlation between the effect of distilled water (Δ_{2,1} as x-axis) and SWC (Δ_{3,2} as y-axis) at the ear area of both left and right SJ21. There was a total of 54 total cases with 39 of those cases having >±0.25 temperature change resulting in 72.2% significance. Positive values mean heating up and negative values mean cooling down. Note: the points that are repeated are shown as only one marker in the graph.

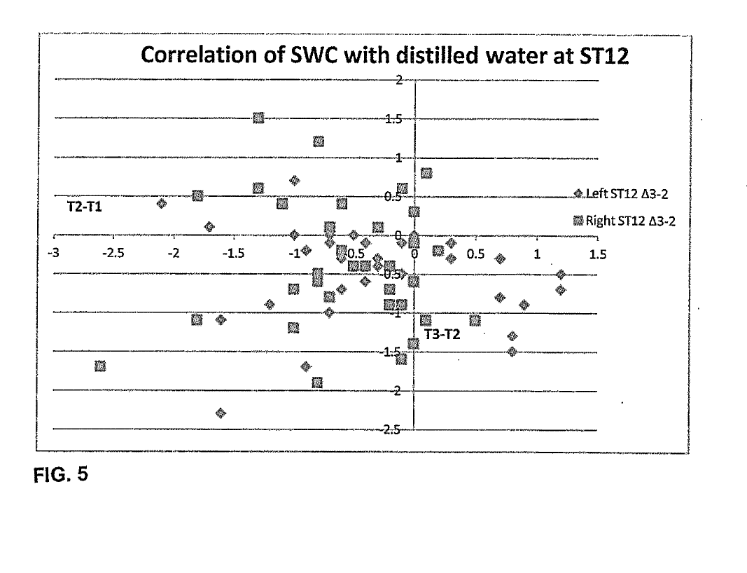

FIG. 5 shows the correlation between the effect of distilled water (Δ₂₁ as x-axis) and SWC (Δ₃₂ as y-axis) at the collar bone area of left and right ST12 both left and right GB 14. There was a total of 58 total cases with 48 of those cases having >±0.25 temperature change resulting in 82.8% significance. Positive values mean heating up, and negative values mean cooling down. Note: the points that are repeated are shown as only one marker in the graph.

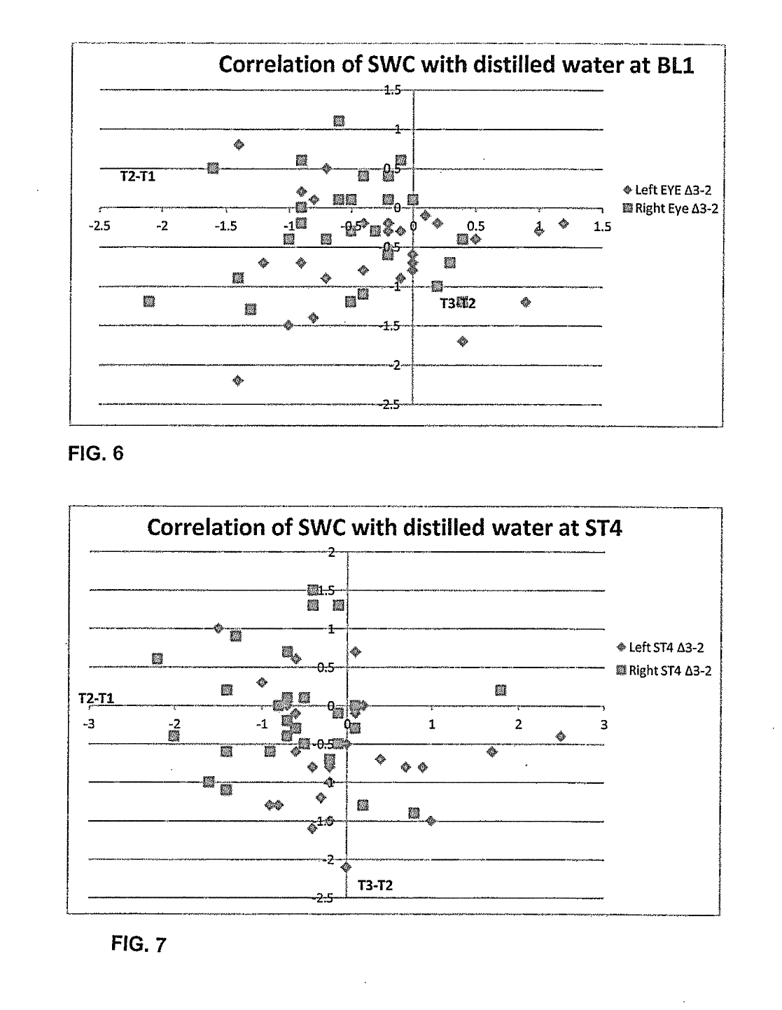

FIG. 6 shows the correlation between the effect of distilled water (Δ₂₁ as x-axis) and SWC (Δ₃₂ as y-axis) at left and right acupoints BL1 of eye areas. There was a total of 60 total cases with 44 of those cases having >±0.25 temperature change resulting in 73.3% significance. Positive values mean heating up and negative values mean cooling down. Note: the points that are repeated are shown as only one marker in the graph.

FIG. 7 shows the correlation between the effect of distilled water (Δ₂₁ as x-axis) and SWC (Δ₃₂ as y-axis) at left and right mouth corners of acupoints ST4. There was a total of 56 total cases with 49 of those cases having >±0.25 temperature change resulting in 87.5% significance. Positive values mean heating up and negative values mean cooling down. Note: the points that are repeated are shown as only one marker in the graph.

FIG. 8 shows the correlation between the effect of distilled water (Δ₂₁ as x-axis) and SWC (Δ₃₂ as y-axis) at ten acupoints: left and right GB14, left and right SJ21, left and right ST4, left and right BL1, and left and right ST12. There was a total of 282 total cases with 225 of those cases having >±0.25 temperature change resulting in 79.8% significance. Note: the points that are repeated are shown as only one marker in the graph. Positive values mean heating up and negative values mean cooling down.

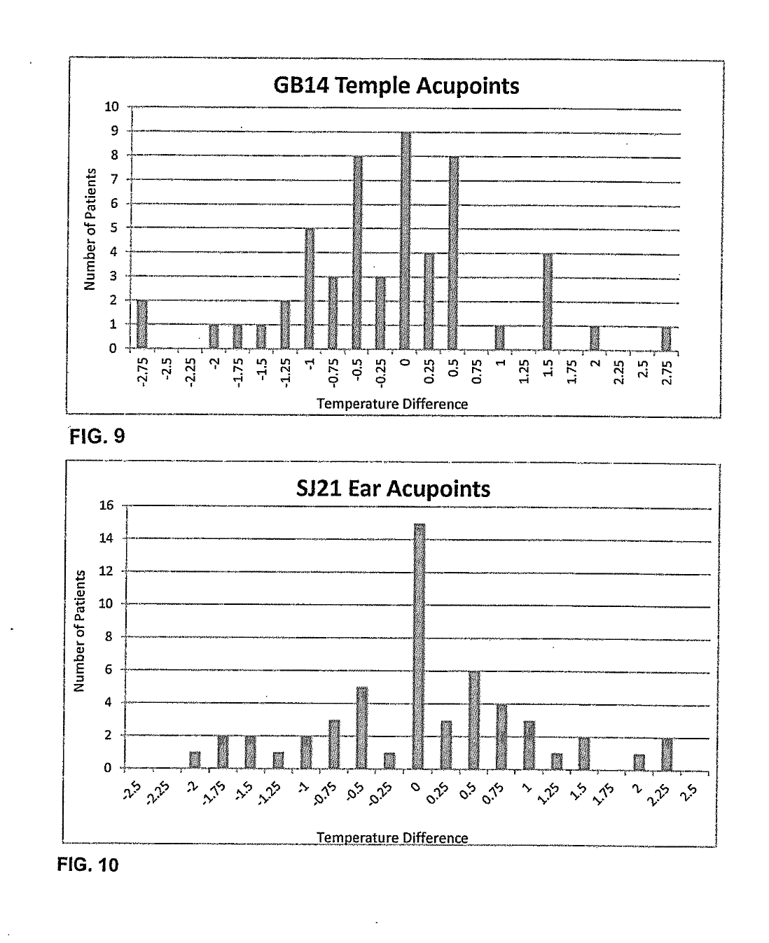

In the following figures below (FIG. 9-14) we showed the quantitative healing effect difference (Δ) of SWC, which was obtained after the subtraction of placebo effect from distilled water. The horizontal axis represented by values of Δ from −2.75° C. to +2.75° C. They were divided into bin of size 0.25° C. In the vertical axis it showed the numbers of subjects who had those particular values of Δ in each bin. We have 30 subjects. Each subject has two acupoints GB 14, two SJ21, two BL1, two ST4 and two ST12. In principle we should have 300 independent hot spots to measure the maximum temperatures. However due to the covering of the hot spots by hair which happened occasionally when pictures were taken, we had only usable 282 points that we could measure maximum temperatures.

In FIG. 9 the results for two GB14 (temples) are displayed. There were 54 cases total with 45 of those having >±0.25° C. temperature change thus resulting in 83.3% (p<0.01), which was a significant temperature change in a period of 15 minutes. The interval of >±0.25° C. was chosen because it is two and a half standard deviation of the statistical fluctuation ±0.1° C. of skin temperature.

In FIG. 10 the results for two SJ21 (ears) are displayed. There were 54 cases total with 39 of those having >±0.25° C. temperature change thus resulting in 72.2% (p<0.01).

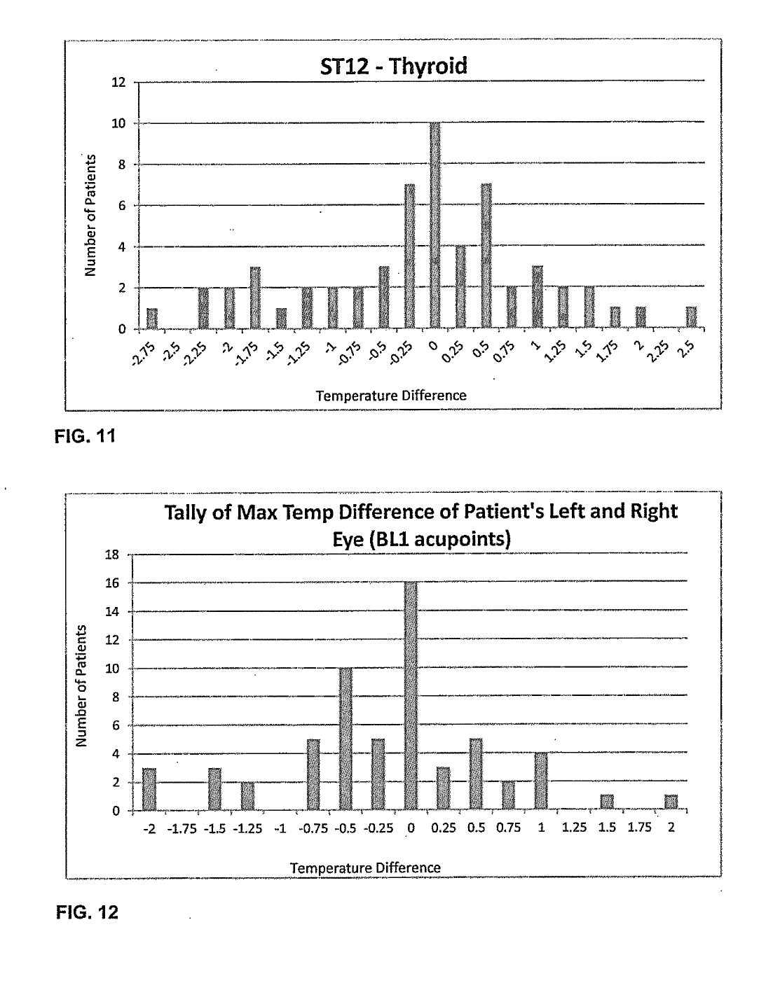

In FIG. 11 the results for two ST12 (collar bones, thyroids) were displayed. There were 58 cases total with 48 of those having >±0.25° C. temperature change thus resulting in 82.8% (p<0.01).

In FIG. 12 the results for two BL1 (eyes) were displayed. There were 44 points out of a total 60 with Δ>±0.25° C. temperature change thus resulting in 73.3% (p<0.01).

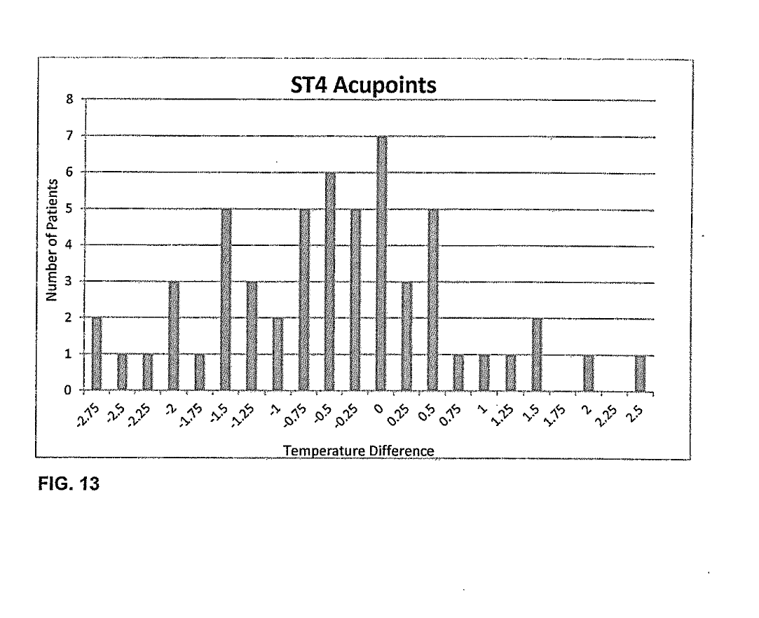

In FIG. 13 the results for two points at ST4 (mouth) are displayed. There were 56 cases total with 49 of those having Δ>±0.25° C. temperature change thus resulting in 87.5% (p<0.01).

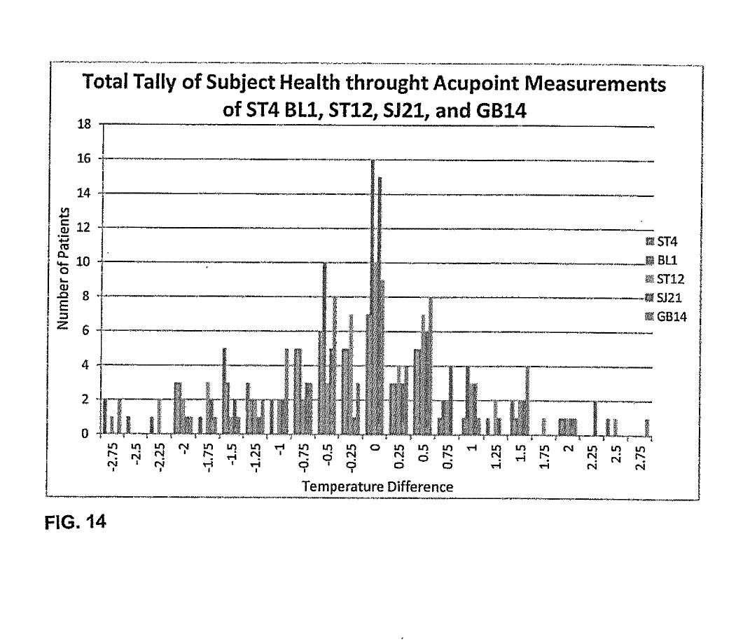

In FIG. 14 the results for all ten points together: two GB14, two SJ21, two BL1, two ST4, and two ST12 are displayed. There were 225 points out of a total 282 with Δ>±0.25° C. temperature change thus resulting 79.8% (p<0.01).

FIG. 9 shows the distribution of the number of independent measurement at left and right GB14 for 30 subjects as a function of the maximum temperature difference Δ with each bin having the size of 0.25° C. There were a total 54 independent points of measurement with 45 of those having >±0.25° C. temperature change resulting in 83.3% significant value.

FIG. 10 shows the distribution of the number of independent measurement at left and right SJ21 for 30 subjects as a function of the maximum temperature difference Δ with each bin having the size of 0.25° C. There were a total 54 independent points of measurement with 39 of those having >±0.25° C. temperature change resulting in 72.2% significant value.

FIG. 11 shows the distribution of the number of independent measurement at left and right ST 12 (collar bone area, thyroids) for 30 subjects as a function of the maximum temperature difference Δ with each bin having the size of 0.25° C. There were a total 58 independent points of measurement with 48 of those having >±0.25° C. temperature change resulting in 82.8% significant value.

FIG. 1 shows the distribution of the number of independent measurement at left and right BL1 (eyes) for 30 subjects as a function of the maximum temperature difference Δ with each bin having the size of 0.25° C. There were a total 60 independent points of measurement with 44 of those having >±0.25° C. temperature change resulting in 73.3% significant value.

FIG. 13 shows the distribution of the number of independent measurement at left and right ST 4 (mouths) for 30 subjects as a function of the maximum temperature difference Δ with each bin having the size of 0.25° C. There were a total 56 independent points of measurement with 49 of those having >±0.25° C. temperature change resulting in 87.5% significant value.

FIG. 14 shows the distribution of the number of independent measurement at ten points: GB14, SJ21, BL1, ST 4, and ST 12 1eft and right ST 4 (mouths) for 30 subjects as a function of the maximum temperature difference Δ with each bin having the size of 0.25° C. There were a total 282 independent points of measurement with 225 of those having >±0.25° C. temperature change resulting in 79.8% significant value.

Methods and results of the long term healing effect of the stable water clusters will be now described in more detail.

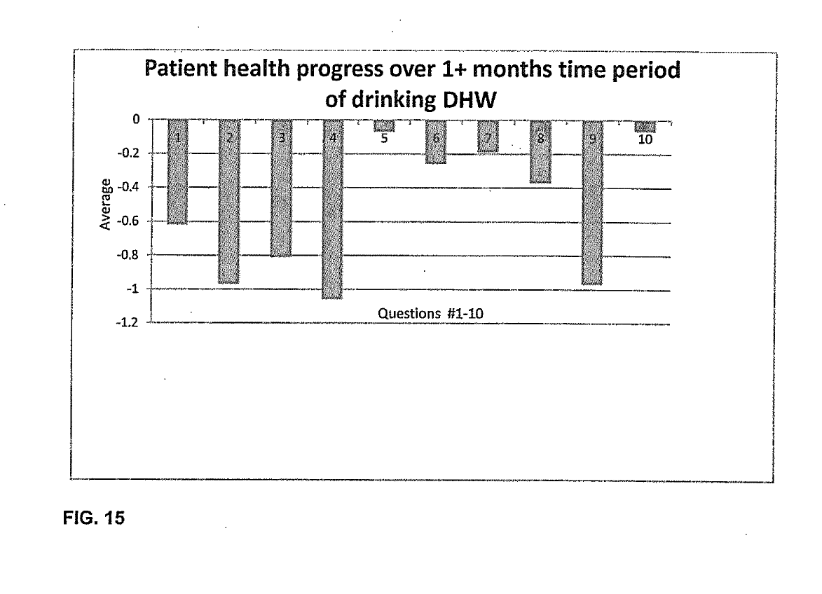

FIG. 15 shows a patient health progress over 1 plus month time period of drinking of stable water clusters in form of a double helix water.

A panel of 31 returning subjects were asked 10 questions regarding general health from 1 (best) to 10 (worst he were asked to fill out questionnaire during initial consult and each time they returned for a follow-up,

1. General health

2. General pain level

3. Ability to sleep

4. Energy Level

5. Circulatory system (heart, liver, arteries)

6. Digestive problems (intstinies, stomach)

7. Respiratory system (lungs)

8. Reproductive organs

9. Concentration (ability to focus, attention span)

10. Memory

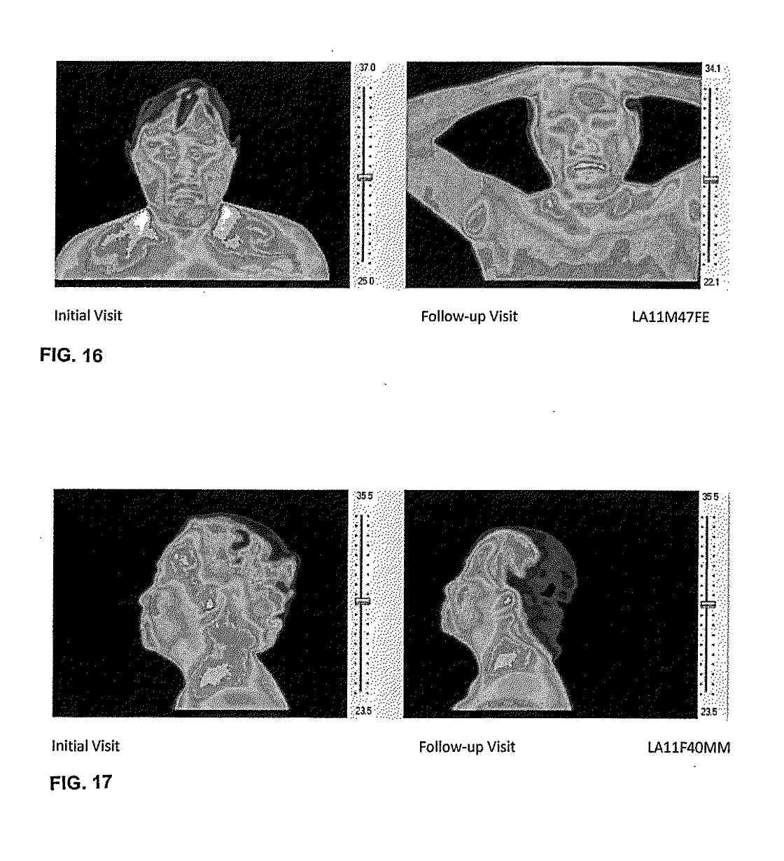

FIGS. 16 and 17 illustrates the thermo images during initial visit and a follow-up visit (left image and right image correspondingly) for two test cases.

Thermo images of a 55+ year old male subject with initial visit (left image) and follow-up visit (right image). Health questionnaire indicates the patient feels an improvement in his general health, circulatory health, respiratory health, and digestive health. Initial visit and follow-up visits were made 1 month apart, during which the subject drank 8oz SWC two times a day. In the initial visit subject has never drank SWC. Both images show patient before drinking SWC for that day.

Thermo images of a 55+ year old female subject with initial visit (left image) and follow-up visit (right image). Health questionnaire indicates the patient feels an improvement in her memory. Initial visit and follow-up visits were made 2 months apart, during which the subject drank 8 oz SWC two times a day. Both images show subject before drinking SWC for that day.

The present invention is not limited to the details shown since various modifications and changes are possible without departing from the spirit of the invention.

What is desired to be protected by Letters Patent is set forth in particular in the appended claims.

What we claim is:

1. A method of enhancing health of a person, comprising the step of administering stable water clusters to the person.

2. The method as defined in claim 1, wherein said administering includes administering the stable water clusters to the person having an autoimmune disease.

3. The method as defined in claim 2, wherein said administering includes administering the stable water clusters to the person having the autoimmune disease selected from the group including arthritis rheumatoid arthritis, lupus, diabetes, cancer, asthma, and allergy.

4. The method as defined in claim 1, wherein said administering includes administering the stable water clusters to the person having pain.

5. The method as defined in claim 1, wherein said administering includes administering the stable water clusters having the pain selected from the group including neck pain, upper back pain, lower back pain, pain in fingers, pain in hands, pain in arms, pain in thighs, pain in abdominal area, pain in stomach, pain in heart, pain from an accident, pain in head, pain in ear, pain in eyes, pain in nose, pain in a cheek, pain in a gum, tooth pain, pain in mouth migraine, and pain in sine.

6. The method of claim 1, wherein said administering includes administering of the stable water clusters to the person having a chronic disease.

7. The method of claim 6, wherein said administering includes administering of the stable water clusters to the person having the chronic disease selected from the group including chronic fatigue syndrome and fibromyalgia.

8. The method of claim 7, wherein said administering includes administering the stable water clusters to the person having a mental disease.

9. The method as defined in claim 8, wherein said administering includes administering of the stable water clusters to the person having the mental disease selected from the group including depression, bipolar disorder, schizophrenia ADHD ADD and ASD.

10. The method as defined in claim 1, wherein said administering includes administering of stable water clusters to a person having a genetic disease from malfunction of a normal DNA.

11. The method as defined in claim 1, wherein said administering includes administering of the stable water clusters to the person who is an athlete for improving his performance and alleviating soreness after workouts.

12. The method as defined in claim 1, wherein said administering includes administering of the stable water clusters to the person who suffers, from overworking, stress and toxins selected from the group including biological, chemical and physical toxins.

13. The method as defined in claim 1, wherein said administering includes administering of the stable water clusters in a manner selected from the group including drinking a solution containing stable water clusters through mouth, swallowing small objects which contain the stable water clusters, putting a toical cream which contains the stable water clusters on skin, breathing the stable water clusters through mouth or nose, putting drops which contain the stable water clusters into eyes, ears or nose, cleaning colon with solution that contains the stable water clusters, eating food that contains the stable water clusters, and injecting solution that contain the stable water clusters into blood vessel.
